(19) United States
(12) Patent Application Publication
(10) Pub. No.: US 2015/0250688 A1
(43) Pub. Date: Sep. 10, 2015

(54) RETROREFLECTIVE NAIL VARNISH

(71) Applicants: Antti Pekka RIEKKINEN, Helsinki (FI); Miika Tuomas BRANDER, Helsinki (FI)

(72) Inventors: Antti Pekka Riekkinen, Helsinki (FI); Miika Tuomas Brander, Helsinki (FI)

(21) Appl. No.: 14/429,786

(22) PCT Filed: Feb. 20, 2013
(86) PCT No.: PCT/FI2013/050193

§ 371 Date: Mar. 20, 2015

(30) Foreign Application Priority Data
Sep. 20, 2012 (FI) U20120154

(51) Int. Cl. A61K 8/02; A61K 8/25; A61K 8/72; A61Q 3/02

(52) U.S. Cl. CPC A61K 8/0245; A61Q 3/02; A61K 8/25; A61K 8/72; A61K 2800/412; A61K 2800/42; A61K 2800/26

(57) ABSTRACT

Disclosed is a nail varnish (2), which includes a liquid base part (2) that turns solid. Characteristically of the nail varnish (2), it further includes retroreflective particles.

### FIELD OF THE INVENTION

The present invention relates to cosmetic products. More specifically, the present invention concerns a nail varnish having retroreflective properties.

### PRIOR ART

Nail varnishes are commonly used for cosmetic decoration of nails. Conventionally, a nail varnish consists of a solvent based varnish to which various compounds are mixed in order to achieve desired properties. Typically, nitrocellulose, for example, may be used as a binder, which is then dissolved in e.g. butyl acetate or ethyl acetate. Alternative arrangements are known, in which the nail varnish comprises a liquid compound which cures upon exposure to UV radiation.

Often nail varnishes may include color pigments for achieving a desired color tone to the nail being varnished. In addition to this, solid particles may be mixed with the nail varnish for enhancing the decorative properties and for achieving various surfaces, such as a glittering or an iridescent surface. Different minerals, such as mica, and glittery metal grains may be used as such particles. Other decorative effects used in nail varnishes have been attained with the use of e.g. fluorescent or UV-reflective pigments.

Additionally, nails can be decorated by attaching various decorative films or small objects, so-called nail jewelry, on the surface of the nail. Attachment to the nail can be carried out using a separate adhesive or by laying the film or the nail jewelry on the varnished nail before the varnish has dried.

### SUMMARY OF THE INVENTION

The object of the present invention is to provide a nail varnish having novel decorative properties, and which can be used like a conventional nail varnish. A nail varnish according to the invention is applied to a desired surface, normally a nail. Typically, the nail polish is applied by brush or by spraying. Often nail varnishes are packed for retail in small bottles having a brush suitable for applying nail varnish integrated on their cap.

Characteristically of a nail varnish according to the present invention, it comprises retroreflective particles for reflecting light substantially back in the light's direction of arrival. The characteristics features of the invention's other embodiments become apparent from the accompanied claims.

### BRIEF DESCRIPTION OF THE DRAWINGS

In the following, the invention is discussed in more detail with reference to the following drawing, wherein:

FIG. 1 illustrates schematically a surface coated with a nail varnish according to an embodiment of the invention.

### DETAILED DESCRIPTION OF THE INVENTION

In the following, a retroreflective particle is used to refer to an object having such optical characteristics that light directed onto it is reflected substantially back towards the arrival direction of the light.

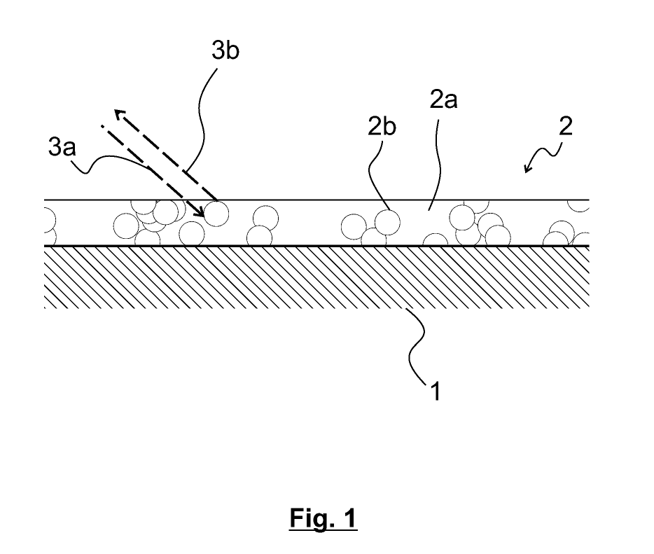

FIG. 1 illustrates a nail 1 which has been coated with a nail varnish 2 according to an embodiment of the invention. For the sake of clarity, retroreflective particles 2b of the nail varnish have been illustrated out of scale. FIG. 1 also shows how light 3a reflects 3b substantially back towards its arrival direction, when directed onto a surface coated with a nail varnish according to an embodiment of the invention.

According to an embodiment of the invention, the nail varnish 2 comprises a liquid base part 2a, which can be made to become solid after the application of the nail varnish 2. The base part 2a may be, for example, a mixture of a binder and a solvent, from which the solvent evaporates as the nail varnish 2 dries. Alternatively, the base part 2a may be, for example, an UV-curable polymer. Retroreflective particles 2b have been mixed to the base part 2a of the nail varnish. In addition to this, the nail varnish 2 may comprise other components for achieving desired properties. Examples of such components are color agents, decorative particles, plasticizers, retardants, film forming agents, resins, solvents, adhesives, thickening agents and UV-stabilizers. In particular, the base part 2a of the nail varnish 2 may include ingredients that can affect the applicability of the nail varnish, such as flowability or levelling. If the base part 2a itself is of a suitable color, it can be regarded as acting as a coloring agent.

A nail varnish according to another embodiment of the invention comprises retroreflective particles 2b preferably 10-50%, more preferably 20-40%, and most preferably 25-35% by volume. A composition of the nail varnish 2 found to be suitable comprises retroreflective particles 33% by volume. In a nail varnish according to yet another embodiment of the invention, the retroreflective particles have a size of preferably 10-100 μm, more preferably 20-80 μm, and most preferably 30-50 μm. Advantageously, the retroreflective particles 2b are of a spherical shape, in which case the sizes mentioned above refer to their diameter. Other shapes and forms of the retroreflective particles 2b may also come in question, given that the retroreflective properties are maintained. In such a case, the sizes of the retroreflective particles 2b mentioned above refer to the greatest dimension of the particle.

Using a suitable amount of retroreflective particles 2b of a suitable size, a highly-reflective appearance can be achieved with the nail varnish 2 while still maintaining good smoothness and quality of a surface.

The retroreflective particles 2b used in a nail varnish 2 according to an embodiment of the invention may be manufactured from a translucent glass or plastics material. In particular, retroreflective particles manufactured from a crystal glass have been found to be suitable in a nail varnish according to the invention. Another composition of a glass material which has been found to be suitable is TiO₂—BaO—SiO₂. Naturally, other materials may be used.

According to an embodiment of the invention, the retroreflective particles 2b of a nail varnish 2 are at least partially coated. Advantageously, highly reflective materials, such as metals, are used for coating. For example, retroreflective particles 2b coated at least partially with aluminum or silver have been found to be suitable in a nail varnish according to the invention. Alternatively, retroreflective particles 2b can be coated at least partially with a material of a certain color tone, e.g. for achieving a similar color tone between the at least partially coated retroreflective particles 2b and the base part 2a. In addition, the retroreflective particles 2b can be at least partially coated with a black material or a material having low reflectivity. By using at least partially coated retroreflective particles as described above, the retroreflective properties of the nail varnish can be enhanced and/or the appearance a the surface treated with the nail varnish can be enhanced in a situation where retroreflection is not in a dominant position with respect to appearance. Such a situation occurs e.g. when a surface coated with a nail varnish according to an embodiment of the invention is observed from a different direction than from which light is projected on it.

In a nail varnish 2 according to another embodiment of the invention, the retroreflective particles 2b reflect light substantially only in one or more wavelength ranges. Advantageously said one or more wavelength range corresponds to a color tone. This can be accomplished by using such retroreflective particles that reflect in one or more wavelength ranges, or alternatively by using one or more different types of retroreflective particles each type reflecting light in a wavelength range of its own. According to still a further embodiment of the invention, the base part 2b of the nail varnish comprises a color agent. The color agent may, for example, be a typical color pigment used in nail varnishes. If the base part 2a has a natural color, it can be considered to contain a coloring agent itself.

Characteristically of a nail varnish according to yet another embodiment of the invention, at least two from the group comprising a coloring agent, retroreflective particles 2b, and the coating of the retroreflective particles 2b, reflect light substantially in one or more common wavelength ranges. Preferably, said one or more common wavelength ranges correspond to a color tone.

Naturally, in a nail varnish according to the invention, different types of retroreflective particles may be used, each of which type corresponding to one or more of the above mentioned embodiments.

According to the invention, retroreflective particles 2b can also be used in connection with other cosmetic products. In such a case, at each time, a suitable quantity of retroreflective particles 2b is mixed with the ingredients forming the cosmetic product. If necessary, the composition of the cosmetic product may be altered in order to adapt the properties of the cosmetic product for use with retroreflective particles.

1. A nail varnish comprising a liquid base part (2a) that turns solid, wherein the nail varnish further comprises retroreflective particles (2b).

2. A nail varnish (2) according to claim 1, wherein the varnish comprises, by volume, 10-50% of retroreflective particles (2b).

3. A nail varnish (2) according to claim 1 wherein said retroreflective particles (2b) have a size of 10-100 μm.

4. A nail varnish (2) according to claim 1, wherein said retroreflective particles (2b) are made of material selected from the group comprising glass materials and plastic materials.

5. A nail varnish (2) according to claim 1, wherein said retroreflective particles (2b) are at least partially coated.

6. A nail varnish (2) according to claim 1, wherein said retroreflective particles (2b) reflect light substantially in one or more wavelength ranges, said one or more wavelength ranges corresponding to a color tone.

7. A nail varnish (2) according to claim 1, wherein the varnish comprises a color agent.

8. A nail varnish (2) according to claim 7, wherein at least two chosen from the group comprising a color agent, retroreflective particles (2b) and the coating of the retroreflective particles, reflect light only in one or more common wavelength ranges, said only one or more common wavelength ranges corresponding to a color tone.

9. A nail varnish (2) according to claim 1, wherein the varnish comprises, by volume, 20-40% of retroreflective particles (2b).

10. A nail varnish (2) according to claim 1, wherein the varnish comprises, by volume, 25-35% of retroreflective particles (2b).

11. A nail varnish (2) according to claim 1 wherein said retroreflective particles (2b) have a size of 28-80 μm.

12. A nail varnish (2) according to claim 1 wherein said retroreflective particles (2b) have a size of 30-50 μm.

13. A nail varnish (2) according to claim 2 wherein said retroreflective particles (2b) are made of material selected from the group comprising glass materials and plastic materials.

14. A nail varnish (2) according to claim 3 wherein said retroreflective particles (2b) are made of material selected from the group comprising glass materials and plastic materials.

15. A nail varnish (2) according to claim 2, wherein said retroreflective particles (2b) are at least partially coated.

16. A nail varnish (2) according to claim 3, wherein said retroreflective particles (2b) are at least partially coated.

17. A nail varnish (2) according to claim 4, wherein said retroreflective particles (2b) are at least partially coated.

18. A nail varnish (2) according to claim 2 wherein said retroreflective particles (2b) reflect light substantially in one or more wavelength ranges, said one or more wavelength ranges corresponding to a color tone.

19. A nail varnish (2) according to claim 3 wherein said retroreflective particles (2b) reflect light substantially in one or more wavelength ranges, said one or more wavelength ranges corresponding to a color tone.

20. A nail varnish (2) according to claim 4 wherein said retroreflective particles (2b) reflect light substantially in one or more wavelength ranges, said one or more wavelength ranges corresponding to a color tone.
